(19) United States
(12) Patent Application Publication
(10) Pub. No.: US 2011/0118541 A1
(43) Pub. Date: May 19, 2011

(54) POSITIONING DEVICE FOR LABORATORY AND MEDICAL DEVICES

(75) Inventor: Jörg Gassmann, Dresden (DE)

(73) Assignee: SAIA-BURGESS DRESDEN GMBH, Dresden (DE)

(21) Appl. No.: 12/621,242

(22) Filed: Nov. 18, 2009

(51) Int. Cl. A61B 1/00; A61M 5/32

(52) U.S. Cl. 600/102; 604/174

(57) ABSTRACT

The invention relates to a poisoning apparatus (1) for laboratory and medical devices, with a support (2) having a positioning field and an actuator coupled with the support (2) and with the laboratory and medical device. The actuator includes a positioning element arranged on the support (2) for rotation or pivoting and having a rotation or pivot axis. For generating a restoring torque acting on the actuator, a bar-shaped or helical spring (6) is provided, which divergent from its helical axis is subjected to both torsion and bending, with the spring extending between the support (2) and the rotatable or pivotable actuator and eliminating or at least reducing the play of the actuator when the medical-technical device is positioned.

### BACKGROUND OF THE INVENTION

(1) Field of the Invention

The invention relates to a positioning apparatus for laboratory and medical devices according to the preamble of claim 1.

(2) Description of Related Art

Positioning apparatuses of this type are frequently employed in laboratories, operating rooms of hospitals and outpatient offices for precisely positioning and temporarily fixing the position of the laboratory and medical devices relative to the test object.

Commercially available positioning apparatuses are supplied, on one hand, as reusable products and, on the other hand, as a single-use products. To ensure a permanent high positioning accuracy, all components of the positioning apparatuses configured as reusable products must therefore be fabricated to exact dimensions. In addition, the drive unit must be coupled to the driven unit without play, because any play contributes extensively to positioning errors. Moreover, reusable products typically include several sensors as well as an associated control unit for position control, which represents a signification cost factor in production. Disadvantageously, positioning apparatuses configured as reusable products must be cleaned—sometimes also sterilized—after each use and therefore require regular service.

Positioning apparatuses configured as single-use products are therefore preferred for various applications, because they can be manufactured at low cost, are easy and fast to install while still having sufficient positioning accuracy.

One document describing the state-of-the-art is WO 2004/021898 A1, in which a guide system for medical devices for the purpose of medical puncturing is described. The guide system includes a support which is positioned on the body of the patient above a defined puncture location. In addition, a needle holder for guiding the needle during puncture as well as a holder for positioning the needle holder relative to the support are provided, wherein the support has a first section which is in sliding contact with a second section that is coupled with the needle holder. This invention is characterized in that both sections cooperate with one another in such a way that the needle holder can be rotatably guided about a point that corresponds to the defined puncture location.

WO 2006/081409 discloses a holder and guide system for medical instruments, in particular biopsy needles. The holder and guide system includes a base support to be positioned on the body of the patient with a window for the puncture location of the biopsy needle and an actuator coupled with the base support, wherein the actuator consists of two mutually orthogonal and independently pivotable arcuate brackets arranged on the support, with each bracket having a pivot axis. A carriage which is movably arranged on one of the two brackets is provided for receiving and fixing the biopsy needle. The carriage also includes means for coupling both brackets to one another. The biopsy needle can be pivoted in all three planes by way of the two pivotable brackets and the carriage.

The aforementioned rotary guide systems or positioning systems have a significant disadvantage that, dependent on the construction of the actuator and the upstream drive unit, a more or less noticeable play is detected in reversing operation. This undesirable play, which is detrimental for medical positioning and fixing of the laboratory and medical devices, significantly contributes to the positioning error of such systems. As a result, laboratory and medical devices configured for example as penetration instruments cannot be inserted into the body of a patient with high precision. The tissue samples to be examined for an early diagnosis of, for example, tumors, which are known to have a very small tissue volume in an early stage, may not be obtained with 100% probability. This frequently leads to misdiagnoses or necessitates additional removal attempts of tissue samples.

It is therefore an object of the invention to propose a positioning apparatus for laboratory and medical devices which can be manufactured at low cost, which can be easily and quickly placed in an operational state and which have a high degree of positioning accuracy.

### BRIEF SUMMARY OF THE INVENTION

According to the concept of the invention, the positioning apparatus for laboratory and medical devices includes a support with a positioning field and at least one actuator coupled with the support and with the laboratory and medical device, with the actuator having at least one positioning element arranged on the support for rotation or pivoting and having a rotation or pivot axis. According to the invention, for generating a restoring torque acting on the actuator, at least one bar-shaped or helical spring is provided, which divergent from its helical axis is subjected to both torsion and bending, with the spring extending between the support and the rotatable or pivotable actuator and eliminating or at least reducing the play of the actuator when the medical-technical device is positioned.

The laboratory and medical devices according to the invention include patient monitoring devices, microscopes, respirators in an intensive care station, computer tomography and nuclear magnetic tomography, dialysis devices, heart pacemakers, infusion pumps, injection pumps, nutrition probes and penetration instruments for medical puncture and endoscopy for human medicine, veterinary medicine and dental medicine.

Medical puncture includes injection of liquid drugs, withdrawal of body fluids and tissues as well as draining of body fluids and gas accumulations. Typically, biopsy needles, injection needles or trocars are used as penetration instruments for puncture.

The positioning element includes two mutually orthogonal arcuate brackets which have each a pivot axis and are arranged on the support so as to be independently pivotable, wherein a bar-shaped or helical spring is associated with each of the brackets in one-to-one correspondence.

The bar-shaped or helical spring according to the invention generates along the entire drive path a torque which depends on the deflection of the actuator configured as positioning element. This arrangement eliminates or at least significantly reduces the play between the coupled components. The bar-shaped or helical spring generates in all operating modes a restoring torque which suppresses the play, without impairing the actuating movement of the drive.

In a first modified embodiment of the invention, the first end of each spring or the helix center, respectively, is attached in an end region of the associated bracket on its pivot axis. The second end of each spring can freely glide on the support in a plane between two end positions during a pivoting motion of the associated bracket, wherein the spring is subjected to torsion and bending.

In a second modified embodiment of the invention, the first end of each spring or the helix center, respectively, is attached in one end region of the associated bracket on its pivot axis. However, the second end of each spring is attached on the support, wherein the spring is subjected to torsion and bending during a pivoting motion of the associated bracket.

Preferably, the spring of the invention has a variable cross-section, preferably a tapered cross-section, from the helix center or from the first end, respectively, to the second end.

Advantageously, the free end or the end of the spring coupled to the support, the respective second end, is formed as a sphere.

The spring characteristic of the bar-shaped or helical spring corresponds here to a combination of the spring characteristic of a conventional helical spring and the spring characteristic of a torsion-bar spring.

According to the second modified embodiment of the invention, the expansion describable by the traveled distance of the free end of the bar-shaped or helical spring is approximately equal to the spring thickness.

To allow low-cost mass production, the bar-shaped or helical spring is produced with a plastic injection molding process. Alternatively, the bar-shaped or helical spring can also be produced by using a so-called 3-D printing process.

In another embodiment of the invention, the positioning element, which is constructed of two mutually orthogonal and independently pivotable arcuate brackets arranged on the support, may have a common spring embodied as spring element. The spring element is here preferably formed of rubber, with the first end of the spring element mounted on the support and the second end on the actuator.

The concept of the invention does not preclude that a separate spring is provided for each bracket of the positioning elements, wherein the spring is formed as torsion-bar spring which extends along the pivot axis between the diametrically opposed end regions of the associated arcuate bracket.

The significant advantages and features of the invention, as compared to the state-of-the-art, are in essence:
Reduction of the play of the positioning apparatus with a bar-shaped or helical spring which extends between the support and the rotatable or pivotable actuator, wherein the spring is subjected to torsion as well as bending divergent from its helical axis,
The bar-shaped or helical spring generates, depending on the deflection of the actuator, along the drive path a restoring torque, which suppresses the play, in all operating states,
To attain a desired spring characteristic curve, the bar-shaped or helical spring has a different cross-section along its length,
A high degree of positioning accuracy is attained in spite of the simple structure and low-cost manufacture,
Simple and quick installation as well as reduced maintenance, and
The positioning apparatus is suitable for different fields of application.

### BRIEF DESCRIPTION OF THE SEVERAL VIEWS OF THE DRAWING(S)

The objects and advantages of the invention can be better understood and evaluated after careful reading of the following detailed description of preferred, but not limiting, exemplary embodiments of the invention in conjunction with the appended drawings, which show in:

FIG. 1 a perspective view of a positioning apparatus in a first preferred embodiment without a laboratory and medical device,

FIG. 2 a perspective view of a positioning apparatus in a first preferred embodiment in cooperation with a laboratory and medical device,

FIG. 3 a cross-sectional view of a bar-shaped or helical spring,

FIG. 4 a perspective view of a positioning apparatus in a second preferred embodiment in cooperation with a laboratory and medical device,

FIG. 5 a perspective view of a positioning apparatus in a third preferred embodiment in cooperation with a laboratory and medical device, and

FIG. 6 a schematic diagram of a different spring characteristic curves.

### DETAILED DESCRIPTION OF THE INVENTION

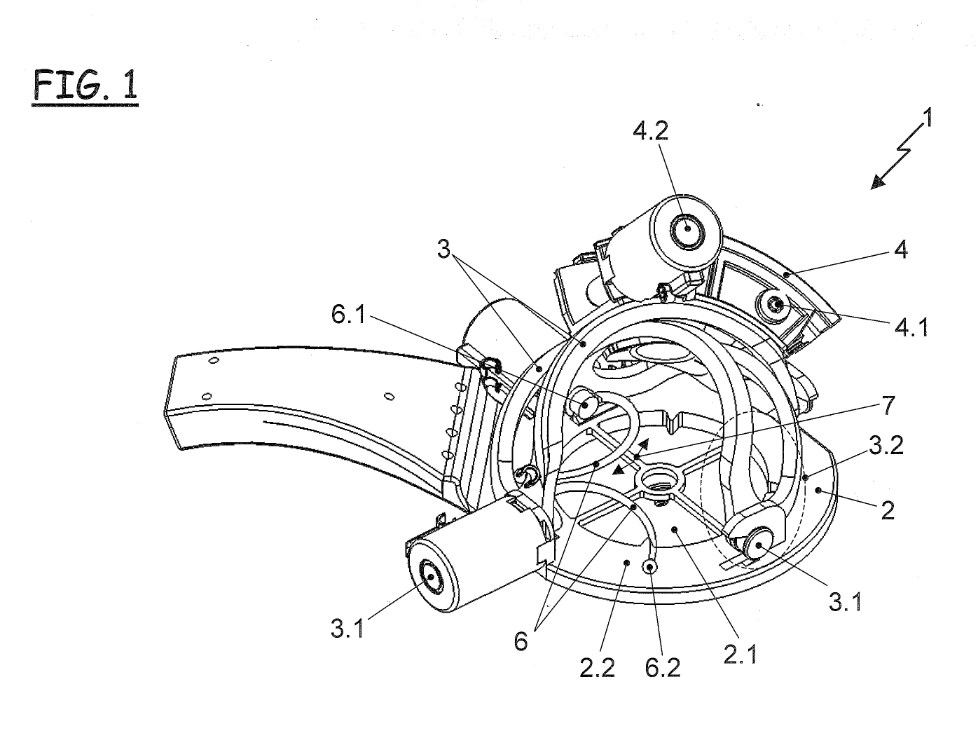

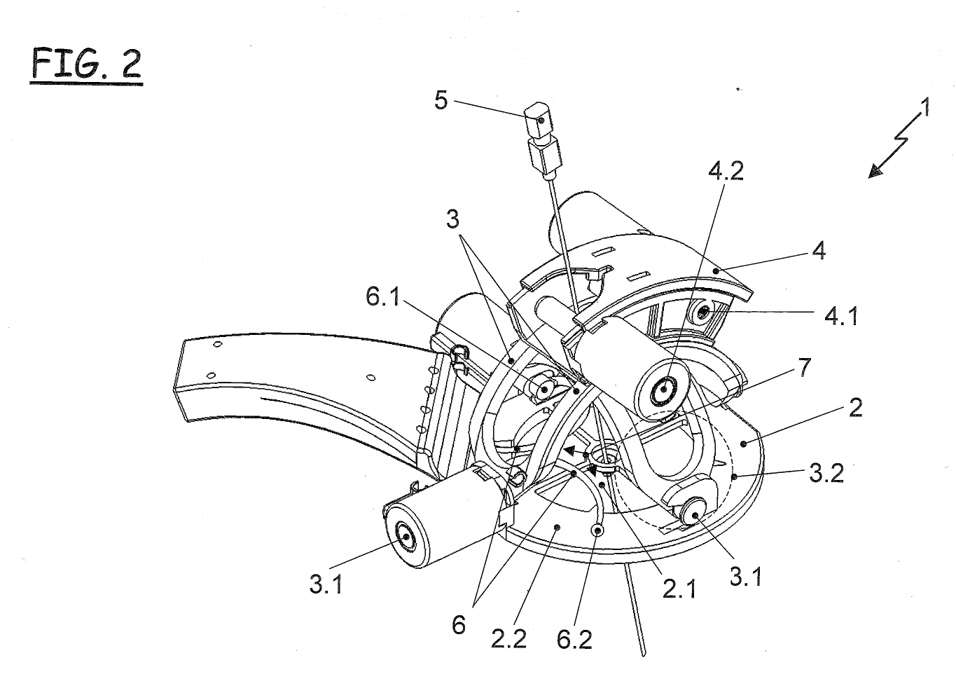

FIG. 1 illustrates in a perspective view a positioning apparatus 1 according to the invention in a first preferred embodiment without a laboratory and medical device. The positioning apparatus for laboratory and medical devices includes a support 2 with a positioning field 2.1 and an actuator coupled with the support 2 and with the laboratory and medical device illustrated in FIG. 2. The actuator has two mutually orthogonal and independent pivotally arcuate brackets 3 arranged on the support 2, with each bracket 3 having a pivot axis 3.1, and a carriage 4 adapted for receiving and fixing a laboratory and medical device, wherein the carriage is movably arranged on one of the two brackets 3. The carriage 4 includes, in addition to a pressure axle 4.1 and a drive shaft 4.2, unillustrated means for coupling the two brackets 3 to one another. The two brackets 3 configured as actuator are each independently controlled by a motor-driven drive. A corresponding bar-shaped or helical spring 6, which can be subjected to both torsion and bending divergent from its helical axis, is provided for generating a restoring torque operating on each pivotable bracket 3. The first end 6.1 of each spring, or the helix center, respectively, is attached in an end region 3.2 of the associated bracket 3 on its pivot axis 3.1 in the region of the drive. Conversely, the second end 6.2 of each spring 6 can freely glide in a plane 2.2 on the support 2 between two end positions during a pivoting motion of the associated bracket 3, wherein each of the two springs is subjected to torsion and bending. The double arrow having the reference symbol 7 points in the direction of the spring expansion in reference to the exemplary spring 6. When the medical device is positioned according to FIG. 2, the play of the actuator, or the play of the two brackets 3, respectively, is eliminated or at least reduced.

FIG. 2 shows in a perspective view the positioning apparatus 1 according to FIG. 1 in cooperation with a laboratory and medical device. The laboratory and medical device is here implemented as a biopsy needle 5 which is held by the carriage 4 and whose needle tip is placed inside the positioning field 2.1 implemented as a crosshair on the subsequent puncture location. The carriage 4 includes a pressure axle 4.1 and a drive shaft 4.2, wherein the biopsy needle 5 is fixed relative to the carriage 4 by the pressure axle 4.1 and advanced with the drive shaft 4.2 towards the positioning field 2.1. Because both brackets 3 can be pivoted independent of one another, the biopsy needle 5 can be suitably tilted for assuming a predetermined position. As a result of the two brackets 3, the biopsy needle 5 can assume any tilt about two axis within a limited cone. The bar-shaped or helical springs 6 associated with the brackets 3 produce a restoring torque that reduces the play of the actuator. The first end 6.1 of each spring, or the helical center, is secured in an end region 3.2 of the associated bracket 3 on its pivot axis 3.1 in the region of the drive. Conversely, the second end 6.2 of each spring can freely glide during a pivot movement of the associated bracket 3 in a plane 2.2 on the carrier 2 between two end positions, wherein each of the two springs 6 is hereby subjected to torsion and bending.

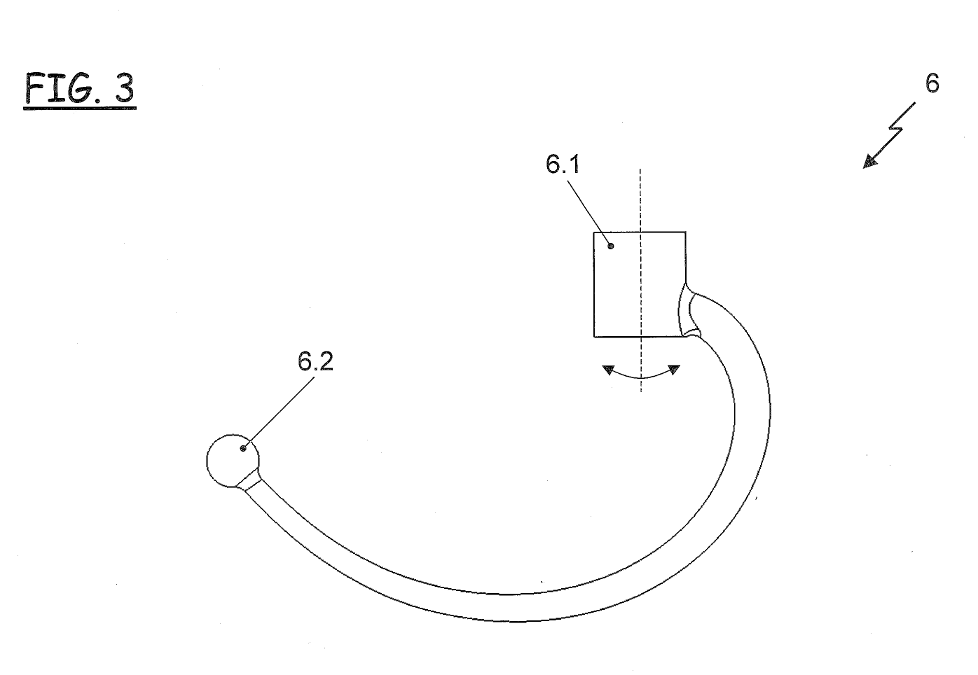

FIG. 3 shows a cross-sectional view of a bar-shaped or helical spring 6, which is subjected to both torsion and bending in a direction divergent from its helical axis. The contour of spring 6 corresponds to the contour of a helical spring of a type employed, for example, in mechanical watches. Unlike with the last mentioned embodiment, the rotation axis is not in the spiral center, but has an orthogonal offset relative to the spiral center. The spring 6, which may be produced using a plastic injection molding process, has a tapered cross-section starting at the helical center, or at the first end 6.1, to the second end 6.2. Each location on the spring 6 is then subjected to the same mechanical stress. According to FIGS. 1 and 2, the first end 6.1 is attached to the pivot axis 3.1 in an end region of the associated bracket 3. During a pivoting motion of the associated bracket 3, the second end 6.2 of each spring 6 can freely glide in a plane 2.2 on the support 2 between two end positions, whereby the spring 6 is subjected to torsion and bending. The second end 6.2 of the spring 6 that is not attached to the pivot axis 3.1 is in the illustrated exemplary embodiment spherical, so that it can easily glide on the support 2. The first end of the bar-shaped or helical spring 6 located on the pivot axis 3.1 of the associated bracket 3 has a cylindrical shape and has a coupling location which is not shown in detail in the drawing. The illustrated double arrow is oriented in the direction of the lengthwise dimensions 7 of the bar-shaped or helical spring 6. The spring characteristic curve of this spring 6 corresponds to a combination of the spring characteristic curve of a conventional helical spring and the spring characteristic curve of a torsion-bar spring. The expansion, which can be described by the travel of the free, second end 6.2 of the bar-shaped or helical spring 6, corresponds approximately to the spring thickness. According to the invention, the expansion of the spring 6 perpendicular to the rotation axis is very small. The spring 6 according to the invention and its operation hence produces a very stiff spring with a helical structure, which cannot be attained in a conventional manner.

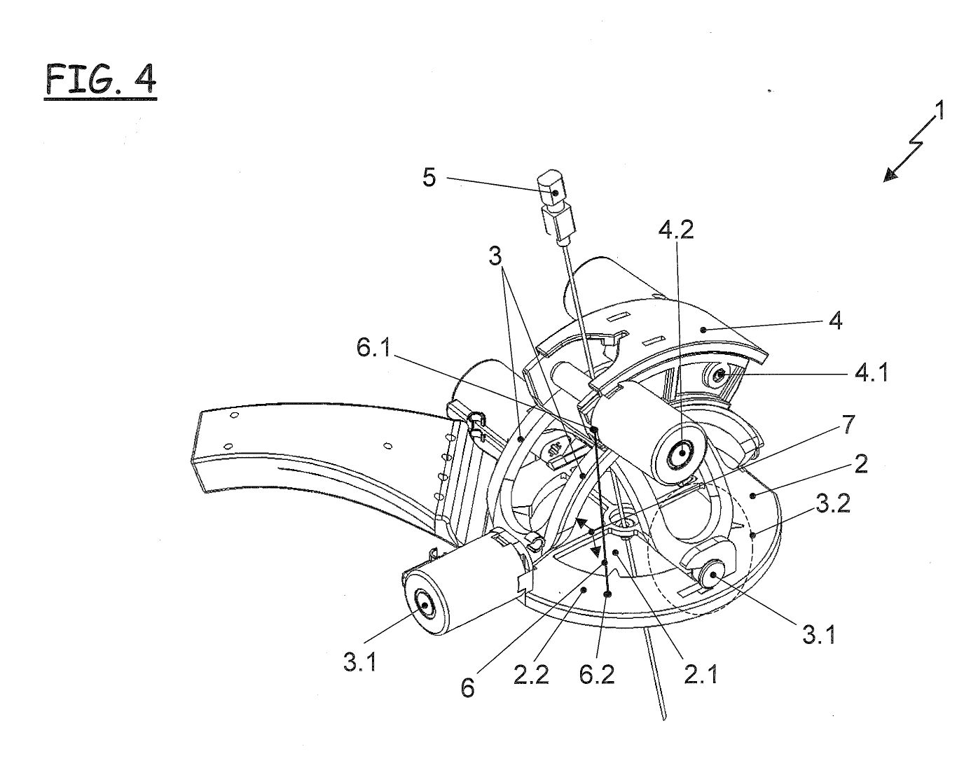

FIG. 4 shows in a perspective view a positioning apparatus 1 in a second embodiment in cooperation with a laboratory and medical device. The basic structure corresponds essentially to that of FIGS. 1 and 2, with the significant difference that instead of two bar-shaped or helical springs 6 only a single spring 6 formed as spring element is employed. The spring element is here implemented as a rubber piece which extends from the carriage 4, which couples the two brackets 3 with one another, to the support 2. A restoring torque operating on the actuator is produced regardless of the position of the two brackets 3 for the purpose of positioning the laboratory and medical device formed as a biopsy needle 5, which reduces the play of the actuator.

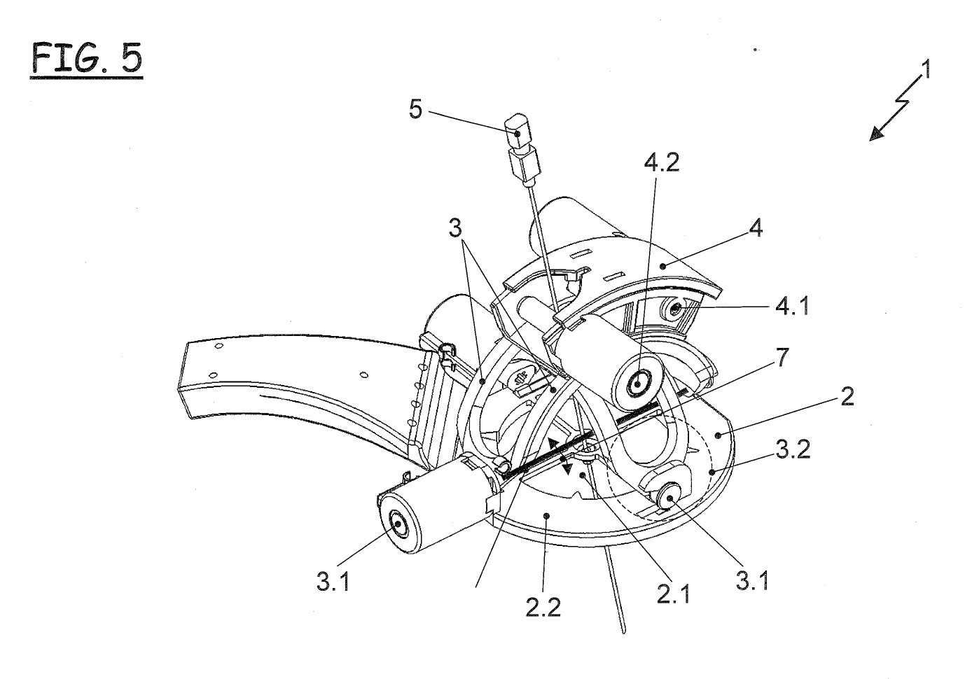

FIG. 5 illustrates in a perspective view a third embodiment of a positioning apparatus 1 cooperating with a laboratory and medical device. The laboratory and medical device is again illustrated as a biopsy needle 5 which is held and positioned by the pressure axle 4.1 and the drive shaft 4.2 of the carriage 4. A dedicated spring 6, which is implemented as a torsion-bar spring, is here provided for each bracket 3 of the positioning apparatus. Each of these two torsion-bar springs extends between the diametrically opposed end regions of its associated arcuate bracket 3 along its respective pivot axis. The positioning elements configured as torsion-bar springs have in the region of the positioning field an unillustrated opening through which the biopsy needle 5 can be freely guided to the puncture location and/or advanced with a drive shaft 4.2.

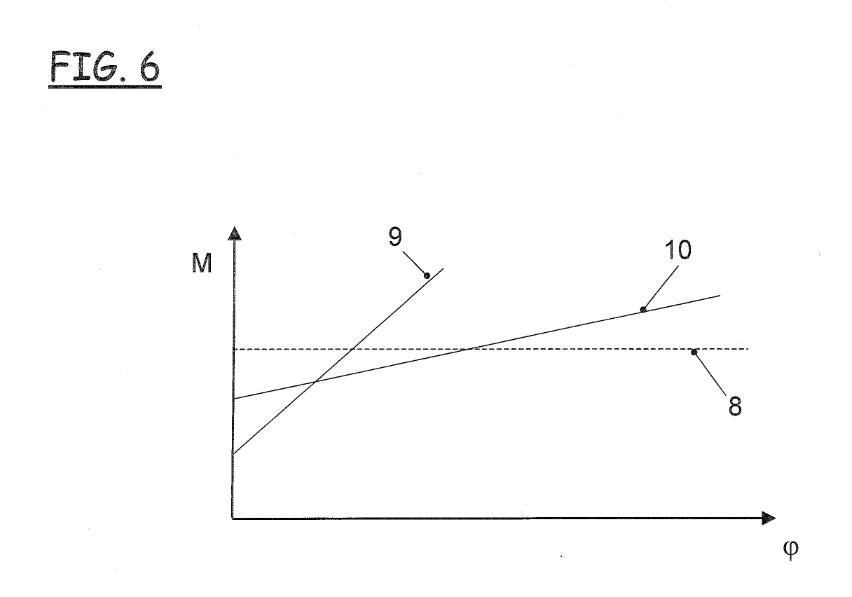

FIG. 6 illustrates in a schematic diagram different spring characteristic curves. The curve identified with the reference symbol 8 corresponds the desired spring characteristic curve 8 with a constant torque. The spring characteristic curve 9 corresponds to the embodiment of the invention, where the first end 6.1 of each spring 6, or the helix center, respectively, is attached in an end region of the associated bracket 3.2 on its pivot axis 3.1, and the second end 6.2 of each spring 6 is attached to the support 2. Both ends 6.1, 6.2 of the spring 6 are therefore clamped; a relatively steep spring characteristic curve 9 is obtained which is significantly different from the desired spring characteristic curve 8. The curves approach the desired spring characteristic curve 8 with constant torque by, as before, attaching the first end 6.1 of each spring 6, or the helix center, in an end region of the associated bracket 3.2 on its pivot axis 3.1, wherein the second end 6.2 of each spring can freely glide in a plane 2.2 on the support 2 between two end positions, wherein the spring 6 is subjected to torsion and bending. The spring characteristic curve indicated with the reference symbol 10 illustrates the last-described embodiment of the invention.

### LIST OF REFERENCE SYMBOLS

1 Positioning apparatus
2 Support
2.1 Positioning field
2.2 Plane
3 Bracket
3.1 Pivot axis of the bracket
3.2 End region of the bracket
4 Carriage
4.1 Pressure axle
4.2 Drive shaft
5 Biopsy needle
6 Spring
6.1 First end of the spring
6.2 Second end of the spring
7 Expansion direction
8 Spring characteristic curve
9 Spring characteristic curve
10 Spring characteristic curve

1. A poisoning apparatus (1) for laboratory and medical devices, comprising
a support (2) with a positioning field and at least one actuator coupled with the support (2) and with the laboratory and medical device, with the actuator comprising at least one positioning element arranged on the support (2) for rotation or pivoting and having a rotation or pivot axis,
wherein, for generating a restoring torque acting on the actuator, at least one bar-shaped or helical spring (6) is provided, which divergent from its helical axis is subjected to both torsion and bending, with the spring extending between the support (2) and the rotatable or pivotable actuator and eliminating or at least reducing the play of the actuator when the medical-technical device is positioned.

2. The positioning apparatus (1) according to claim 1, wherein the laboratory and medical devices are constructed as penetration instruments for medical puncture and endoscopy.

3. The positioning apparatus (1) according to claim 1, wherein the positioning element is comprised of two mutually orthogonal arcuate brackets (3) which have each a pivot axis (3.1) and are arranged on the support (2) so as to be independently pivotable, wherein a spring (6) is associated with each of the brackets (3) in one-to-one correspondence.

4. The positioning apparatus (1) according to claim 1, wherein the first end (6.1) of each spring (6) or the helix center, respectively, is attached in an end region of the associated bracket (3) on its pivot axis (3.1), and the second end (6.2) of each spring (6) freely glides on the support (2) in a plane (2.2) between two end positions during a pivoting motion of the associated bracket (3), wherein the spring (6) is subjected to torsion and bending.

5. The positioning apparatus (1) according to claim 1, wherein the first end (6.1) of each spring (6) or the helix center, respectively, is attached in one end region of the associated bracket (3.2) on its pivot axis (3.1), and the second end (6.2) of each spring (6) is attached on the support (2), wherein the spring (6) is subjected to torsion and bending during a pivoting motion of the associated bracket (3).

6. The positioning apparatus (1) according to claim 1, wherein the spring (6) has a variable cross-section, preferably a tapered cross-section, from the helix center or from the first end (6.1), respectively, to the second end (6.2).

7. The positioning apparatus (1) according to claim 1, wherein the free end or the end of the spring (6.2) coupled to the support is formed as a sphere.

8. The positioning apparatus (1) according to claim 1, wherein the spring characteristic of the spring (6) corresponds to a combination of the spring characteristic of a conventional helical spring and the spring characteristic of a torsion-bar spring.

9. The positioning apparatus (1) according to claim 4, wherein the expansion describable by the traveled distance of the free end (6.2) of the transversely loaded helical spring (6) is approximately equal to the spring thickness.

10. The positioning apparatus (1) according to claim 1, wherein the spring (6) can be produced by using a plastic injection molding process.
